(19) United States
(12) Patent Application Publication
(10) Pub. No.: US 2011/0167009 A1
(43) Pub. Date: Jul. 7, 2011

(54) CONTROLLED-VENDOR MANUFACTURING METHODS

(75) Inventors: Todd H. Klump, Kirkland, WA (US); Dock A. Brown, Bothell, WA (US); John A. Manthey, Stanwood, WA (US); Oscar Hernandez Rojas, Bothell, WA (US); Reza Sharif, Lake Forest Park, WA (US); Xiaodong Zou, Bothell, WA (US)

(73) Assignee: Physio-Control, Inc., Minneapolis, MN (US)

(21) Appl. No.: 12/683,368

(22) Filed: Jan. 6, 2010

(51) Int. Cl. G06Q 50/00; A61B 19/00; G06Q 10/00; G06F 19/00

(52) U.S. Cl. 705/317; 128/897; 700/107; 700/103

(57) ABSTRACT

Embodiments of the invention are directed to manufacturing a medical device in a way that minimizes the possibility of defects. Because of the general increasing desire to eliminate lead from the environment, electronic producers are removing lead from their manufacturing processes. The lead had beneficial qualities, however, in that it prevented other metals, in particular tin, from developing “whiskers,” believed to be caused from thermal and mechanical stress of the tin parts or components. Removing the lead has caused an increasing incidence of failure in electronic devices. Many vendors, believing that lead-free devices are universally desirable, routinely substitute lead-free components for components that previously contained lead. Oftentimes these substitutions are made without knowledge of the buyer. Some devices, in particular life-saving devices, may be adversely affected by such a substitution. Embodiments of the invention prevent non-specified goods from being assembled into the medical device by generating evidence or requiring that vendors generate evidence of component composition for particular components that may be at risk for premature failure in the medical device.

### FIELD OF THE INVENTION

This disclosure relates generally to manufacturing, and, more particularly to methods for manufacturing reliable medical devices by controlling vendor shipments of components or subassemblies.

### BACKGROUND

Reducing the amount of dangerous or hazardous materials in our environment is a laudable goal. Some industries generate more of these materials than others, of course. For example, the electronics industry has long used the material Lead as a common material for producing low-cost electronic parts, due to special qualities of Lead or Lead mixtures such as a low melting point, malleability, durability, and the fact that Lead is electrically conductive. Lead is also a significant component of Tin-Lead solder, a very common solder used for producing electrical components such as printed circuit boards.

Unfortunately, Lead is a hazardous substance and oftentimes leaches into the environment from improperly disposed electronic devices. In response to this problem, there is a world-wide effort to reduce the amount of Lead and other hazardous substances in electronic devices.

As an alternative to Lead, many producers have created some component structures, such as pads and other connections, out of Tin, solely, rather than the Tin-Lead mixture. Using Tin-only structures created new problems. Specifically, when Tin is used without Lead, some Tin structures produce “whisker” defects that increase in severity over time. Metal whiskers are a metallurgical crystalline phenomenon where filiform or spiky metal “hairs” grow from the metal surface. Tin whiskers are believed to occur when the underlying Tin structure relieves internal crystalline stresses, such as thermal stresses, which are exacerbated by high temperature and high humidity. Tin whiskers are especially problematic in components having small inter-structure distances. With reference to FIG. 1, a component 100 includes a central electronic circuit 102 and Tin pads 104. As illustrated in the FIG. 1, metal whiskers 110 are growing and extending from the Tin pads 104. In some cases these Tin whiskers 110 may extend from the Tin pads 104 so far that they touch Tin whiskers growing from neighboring pads. Since Tin Whiskers 110 are conductive, they could cause an electrical short between their respective pads. One of the nefarious problems with Tin whiskers is that the whiskers grow over time. This means that a device that passed all initial production tests may fail during its useful life, because the whiskers hadn't grown before the production tests. An electric short in a consumer product, such as an MP3 player, can cause frustration in the user who expects his or her device to operate properly. Significantly more serious is an electrical short in a medical life-saving device, which can leave the device unable to perform its life-saving function.

Embodiments of the invention address these and other limitations of the prior art.

### BRIEF DESCRIPTION OF THE DRAWINGS

FIG. 1 is a scaled up view of a portion of a faulty electrical device according to the prior art.

FIG. 2 is a block diagram of an example medical device manufactured using methods according to embodiments of the invention.

FIG. 3 is a block diagram illustrating the origin of components and subassemblies used to manufacture the medical device of FIG. 2.

FIG. 4 is a flow diagram illustrating example methods according to embodiments of the invention.

FIG. 5 is a flow diagram illustrating examples of the methods of FIG. 4 that can be used by the manufacturer of the medical device of FIG. 2, to determine which of the components and subassemblies illustrated in FIG. 3 may have the highest likelihood of failure.

FIG. 6 is a flow diagram illustrating example methods according to embodiments of the invention.

FIG. 7 is a block diagram illustrating various example medical device manufacturer acceptance levels of flex circuits incorporated into the medical device, according to embodiments of the invention.

FIGS. 8A and 8B together form a block diagram illustrating various example medical device manufacturer acceptance levels of passive components incorporated into the medical device, according to embodiments of the invention.

FIGS. 9A and 9B together form a block diagram illustrating various example medical device manufacturer acceptance levels of semiconductor components incorporated into the medical device, according to embodiments of the invention.

FIGS. 10A and 10B together form a block diagram illustrating various example medical device manufacturer acceptance levels of connector components incorporated into the medical device, according to embodiments of the invention.

FIG. 11 is a diagram of an example test report used by the medical device manufacturer to determine whether particular lots of components or subassemblies should be included in the medical device of FIG. 2.

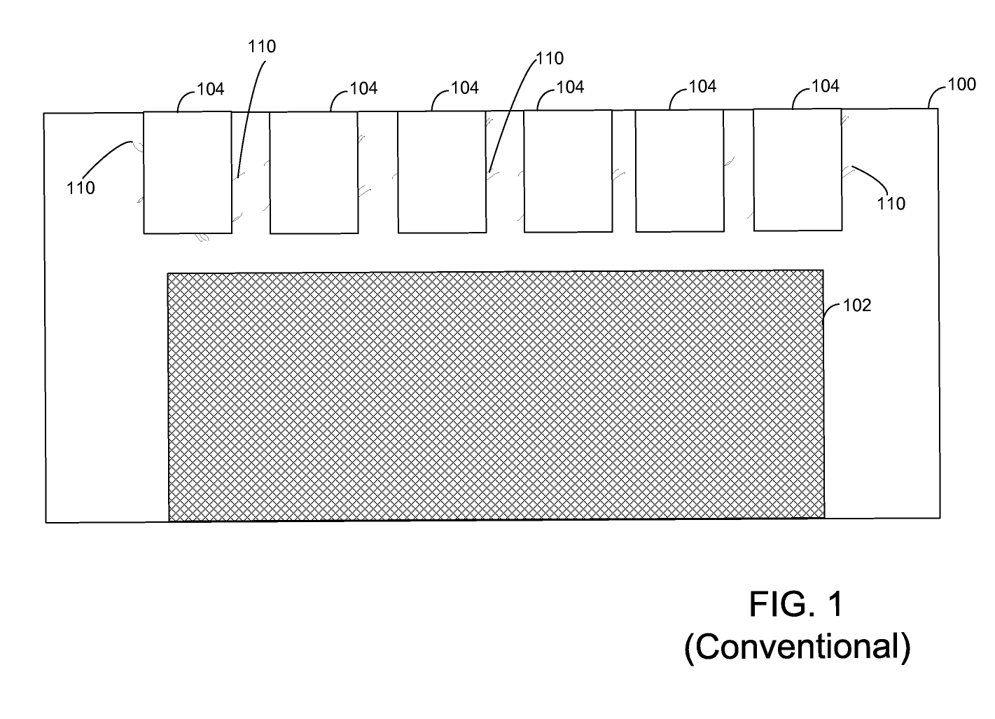

### DETAILED DESCRIPTION

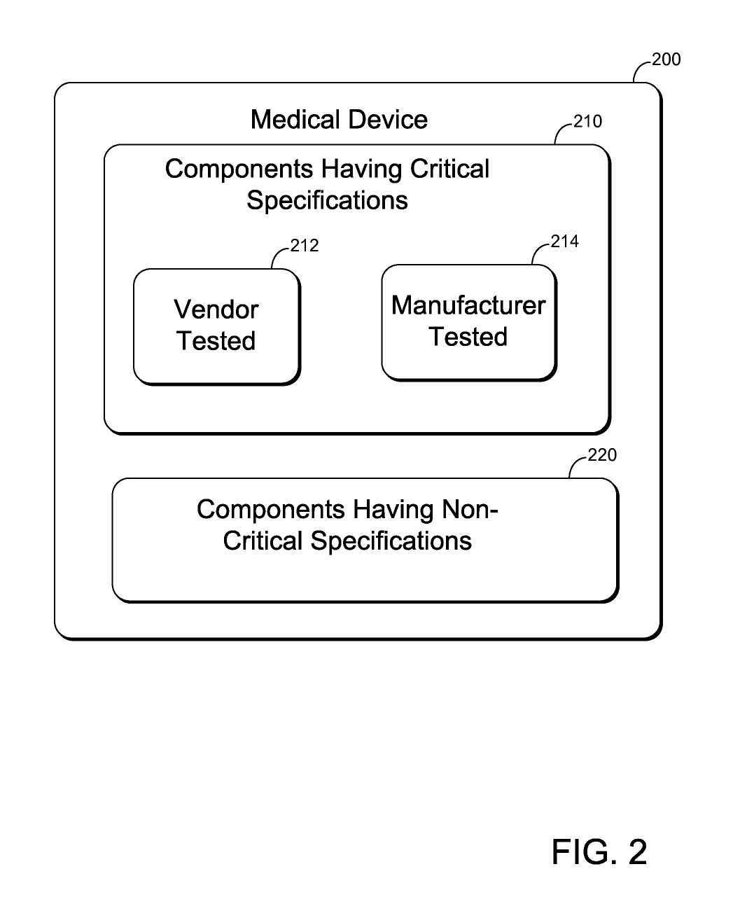

FIG. 2 is a block diagram of an example medical device manufactured using methods according to embodiments of the invention. In FIG. 2, a physical product, such as a medical device 200 is illustrated. The medical device 200 is made from several components, of course, which may be categorized and described in a number of ways. In general, the medical device 200 in this illustration includes components that have critical specifications 210 as well as components that have non-critical specifications 220. Critical specifications include those qualities of a component or assembly that indicate a higher likelihood of failure during the life of the product, as described in detail below.

For components having critical specifications 210, further categorizations may exist. Typically when a manufacturer orders a component or sub-assembly from a vendor or supplier, the specific component is selected based on published specifications by the vendor. For example, a vendor that sells batteries may publish specifications about the batteries, so that a manufacturer can select the correct product to meet the manufacturer's needs. The battery specifications may include, for example, material type, physical size, voltage, storage capacity, and the number of cycles that can be recharged. The manufacturer selects the appropriate battery, based on the specifications, then assembles the selected battery into the manufactured product.

One problem with published specifications is that they may not match the actual specifications of their component product. This is especially true when the component product is manufactured differently than when the specifications were generated. In the battery example given above, the battery producer may change raw material suppliers to achieve a better price, which may have a detrimental effect on the number of recharging cycles that the new batter can withstand. Unless the vendor tests every specification every time a production change is made, the specifications may, in fact, not match the actual performance of the product as delivered. In this instance, the actual battery shipped under the old specifications may not meet the specified number of recharge cycles. Usually a vendor rates its components at “minimum” specifications, with a conservative minimum number that the vast majority, if not all, of the components should meet. For example, if a battery producer tests the number of recharge cycles for a significant number of batteries in a specific lot of batteries, and the lowest number of recharge cycles was 1581, a vendor of the batteries may state in the published specifications that the maximum number of recharge cycles is 1500.

As described above, Lead is gradually being removed as a material used in the production of electronic components and devices. Generally the vendors of such devices will advertise or publish the fact that their components are made without Lead. There is an industry reluctance, however, to change part numbers of components. Thus, a manufacturer who receives components or assemblies from a vendor may not receive specific notice that the producer has changed an underlying component, such as a part number change, absent the manufacturer searching for a published notice to that effect. In other words, a manufacturer may not be informed that a producer or vendor has substituted a component or sub-assembly that previously contained Lead with a newer, Lead-free part, because the vendor may not change the component part number but only change the part specifications. Because most electrical products include a multitude of parts, and because it is unlikely that the manufacturer is continuously researching the vendor specifications for each of the multitude of parts, the manufacturer may never know that a component or part no longer contains Lead. This is especially true because most vendors consider a Lead-free product to at least equal in form, fit, and function to a product that contains Lead, and generally believe that all manufacturers would rather receive the Lead-free part. Thus, in some cases, vendors substitute Lead-free parts for parts that previously contained Lead without notifying their own customers. As described above, however, substituting Tin parts for parts that previously contained a Tin-Lead mixture can lead to failure of a device manufactured with the Tin parts.

Therefore, embodiments of the invention include an inspection step inserted into the manufacturing process. Specifically, for components having critical specifications, described below, embodiments of the invention include a process where such critical components are tested, either by the medical device manufacturer or by the vendor to ensure that the components meet the manufacturer's specifications, without regard to the published specifications of the components. With reference back to FIG. 2, the components having critical specifications 210 are further categorized into those components that are vendor tested 212, and those components 214 that are tested by the manufacturer of the medical device 214.

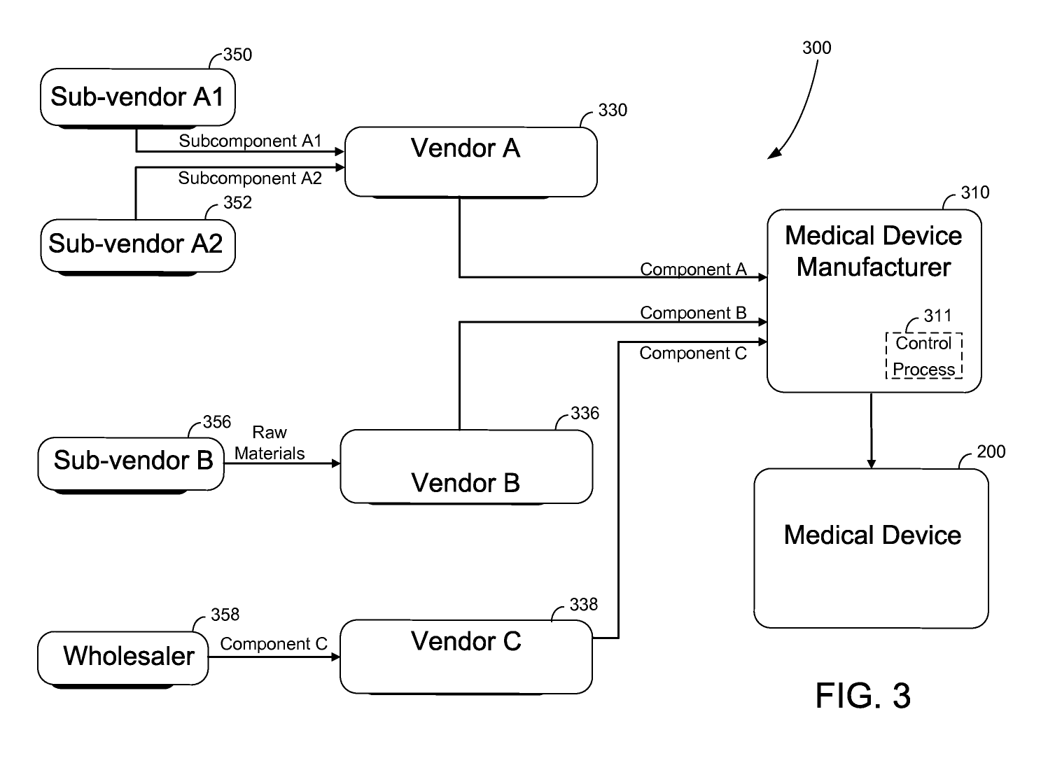

FIG. 3 is a block diagram illustrating the origin of components and subassemblies used to manufacture the medical device 200 of FIG. 2. A medical device manufacturer 310 creates, produces, and assembles the medical device 200 using a variety of materials, components, and sub-assemblies that are produced by a vendor or sub-vendor. The medical device manufacturer is the final assembler of the medical device 200. Each of these parts of the medical device is referred to herein and illustrated in FIG. 3 as a “component,” regardless of whether the part is a single part, such as a resistor or capacitor, a component, such as a voltage rectifying circuit, or a sub-assembly such as a paddle having integrated sensors.

In this simplified illustration of FIG. 3, the medical device manufacturer 310 receives component A from vendor A 330, receives component B from vendor B 336, and receives component C directly from vendor C 338. In turn, each of the vendors 330, 336, and 338 receives a product from another party before delivering it to the medical device manufacturer 310. For instance, the vendor A 330 receives subcomponents A1 and A2 from sub-vendors A1 350 and A2 352, respectively. The vendor A 330 creates the component A from these subcomponents A1 and A2. Differently, the vendor B 336 receives a shipment of raw materials from its sub-vendor B 356. The vendor B 336 then produces component B and passes it to the medical device manufacturer 310. Finally, vendor C 338 simply resells component C, which vendor C received from a wholesaler 358. Each of these delivery paths of components A, B, and C represents different ways that the medical device manufacturer 310 can receive components for its medical device. Vendors A 330, B 336, and C 338 are sometimes termed “Tier-1” or “first-tier” vendors or suppliers because they have a direct relationship with the medical device manufacturer 310. Sub-vendors A1 350, A2 352, B 356, and the wholesaler 358 are sometimes termed “Tier-2” or “second-tier” vendors or suppliers because they have a direct relationship with a Tier-1 vendor, but do not have a direct relationship with the medical device manufacturer 310 itself

The diagram of FIG. 3 also illustrates how difficult it is for the medical device manufacturer 310 to track the provenance of each component that ultimately makes up the medical device 200. For instance, if sub-vendor A2 352 changes one of its own sub-suppliers (not illustrated), the medical device manufacturer 310 may never know that the specifications of component A may have likewise changed. Thus, a level of distrust in the vendors and sub-vendors of the medical device manufacturer 310 can increase in magnitude, commensurately with the distance from manufacturer 310.

Embodiments of the invention take steps to ensure that components originally selected by a medical device manufacturer 310 are not substituted with unsatisfactory components. A process 311 is performed by the medical device manufacturer 310, the details of which are given below, to ensure that only desirable components and sub-assemblies are used in the medical device 200.

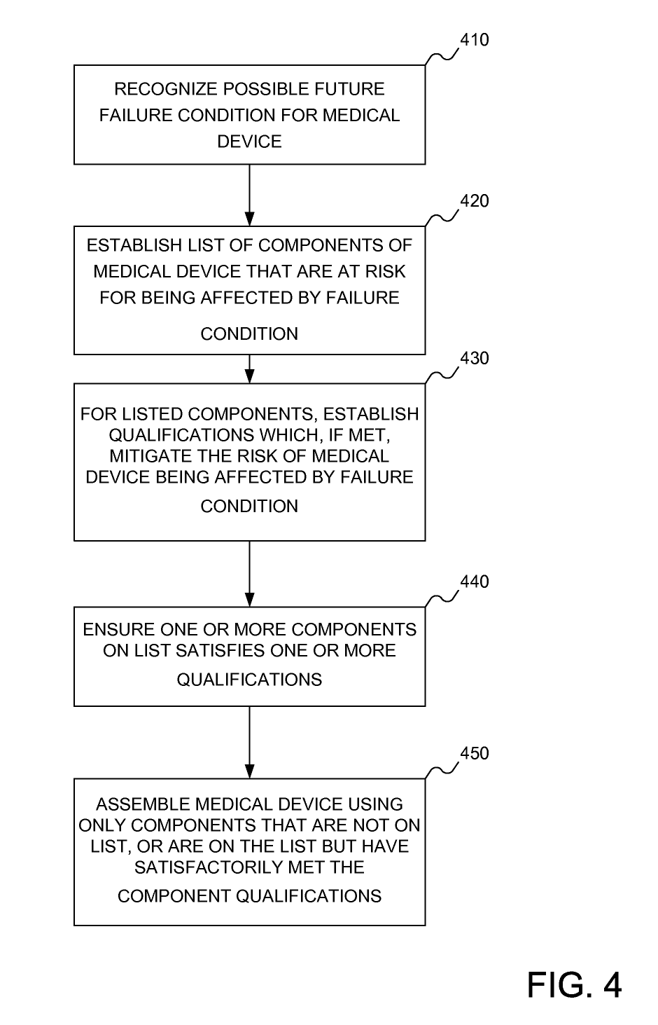

With reference to FIG. 4, a flow diagram illustrates example processes used in embodiments of the invention. In FIG. 4, a medical device manufacturer recognizes that a particular failure condition in a medical device is possible in a process 410. For example, as described above, the medical device manufacturer may recognize that Tin whiskers can grow over time from component structures, creating a short with neighboring structures, and ultimately cause the medical device to fail its critical function. To prevent such failure from occurring, a list of components of the medical device that are at risk for failure is compiled in a process 420. Creating such a list depends on the nature of the failure condition established in the process 410. For example, with regard to the failure condition caused by Tin whiskering, creating the list of at risk components includes creating a list of components having small structure-to-structure distances. This is because Tin whiskers are known to span small gaps between Tin structures, but are less likely to span large gaps.

In the process 430, a particular gap distance is determined by the medical device manufacturer for each of the at-risk components on the list created in the process 420. Different component types on the list made in the process 430 may have different gap distances. For example a semiconductor component may have a particular critical gap distance while connector components have a different critical gap distance. Thus, in the process 430, particular qualifications are created for each type of component on the list created in the process 420. Further, there may be multiple qualifications for each type of component. Thus, a medical device manufacturer may specify that semiconductor components having an air gap of greater than 0.35 mm are acceptable if made out of a first material, but not acceptable if made out of a second material. Detailed examples appear below. The list created as a result of the process 420 is termed a “watch list” because the components on the list are watched by the medical device manufacturer to be sure that they satisfy their minimum performance requirements.

In the process 440, the components on the watch list created in the process 420 are tested to be sure that each component satisfies the minimum component requirements set by the medical device manufacturer. In some instances the testing may be performed by the medical device manufacturer. In other instances the vendor may perform the test, then generate data or evidence for the medical device manufacturer that proves, to the medical device manufacturer's satisfaction, that the components pass the standards set by the medical device manufacturer. The tests may be sample or lot tests only, such as testing two components out of a shipment of five-hundred, or one in one-hundred.

Finally, in a process 450, the medical device is assembled using only those components that passed the test performed in the process 440, or those components that were not on the list of at-risk components. Thus, manufacturing a medical device using these processes minimizes the risk that the medical device will fail for the failure condition for which the tests were established.

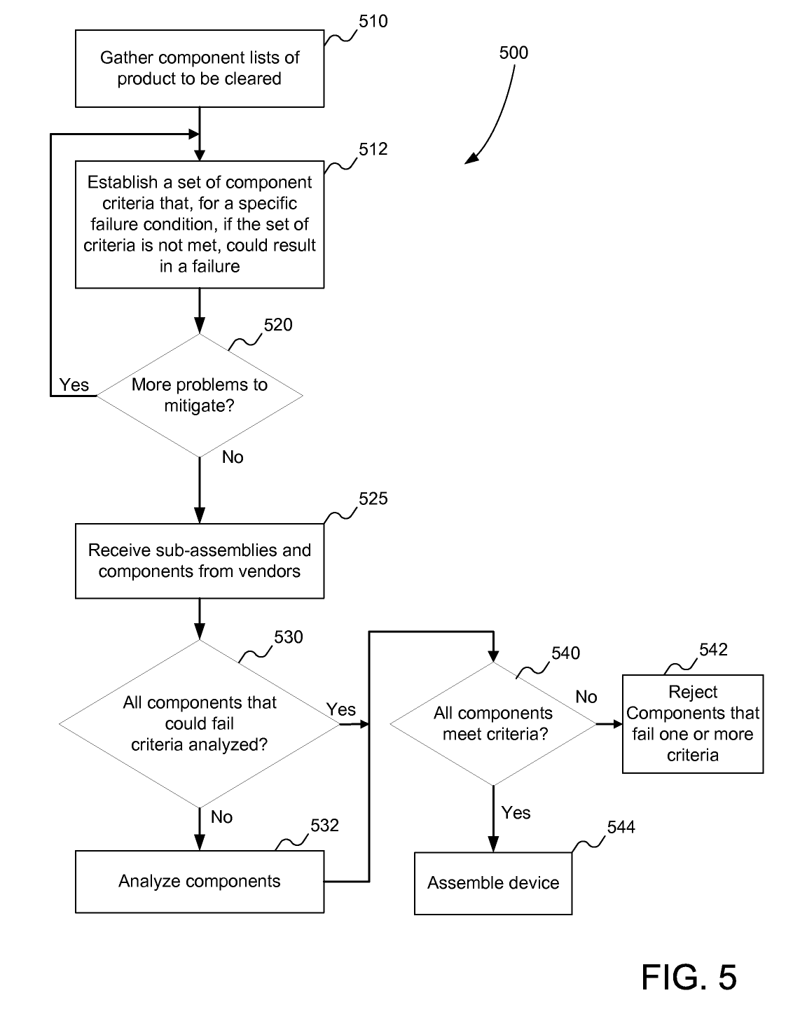

FIG. 5 is a flow diagram illustrating examples of the methods of FIG. 4 that can be used by the manufacturer of the medical device of FIG. 2, to determine which of the components and subassemblies illustrated in FIG. 3 may have the highest likelihood of failure. FIG. 5 includes additional detail about embodiments of the invention. Similar to the processes of FIG. 4, the flow 500 of FIG. 5 begins with gathering or producing component lists of the components making a particular medical device in a process 510. A process 512 refines the list generated in the process 510 by including conditions that, if met, would satisfy the medical device manufacturer that such components could be used in the medical device and not substantially increase the likelihood of failure. The process 520 ensures that each failure condition is separately considered, for medical devices that have more than one failure condition.

In a process 525, the sub-assemblies and components are received from vendors. A process 530 determines if all components that could fail have been analyzed against the criteria, which for the first time through is necessarily exited in the NO direction. When the process 530 is exited in the NO direction, the components are analyzed in a process 532. As described above, the tests can be performed by the medical device manufacturer or by the vendor of the particular component. Specific to the Tin whisker fail condition, one method to implement the testing process 532 is to examine metallurgical makeup of the metal structures within the components. One method to determine metal content is to use X-Ray Fluorescence (XRF), which uses spectroscopy. An XRF analyzer typically generates a numerical display that can be read by the operator to ensure that the metal is made from at least (or less than) a certain percentage of the measured component. Another test method is to use a Scanning Electron Microscope, but may not be preferred because it is relatively more expensive than using XRF.

A process 540 determines if all of the components have been checked against the criteria established in the process 512. If some of the components did not pass the analysis, they are rejected in the process 542. Rejection can take the form of instructing a vendor to not ship a component that cannot pass the criteria, or refusing to accept delivery of a component that fails a criteria analysis. In some embodiments only certain components of a larger shipment may be spot checked for criteria compliance. After all of the components have satisfactorily passed the analysis, the medical device is assembled in the process 544.

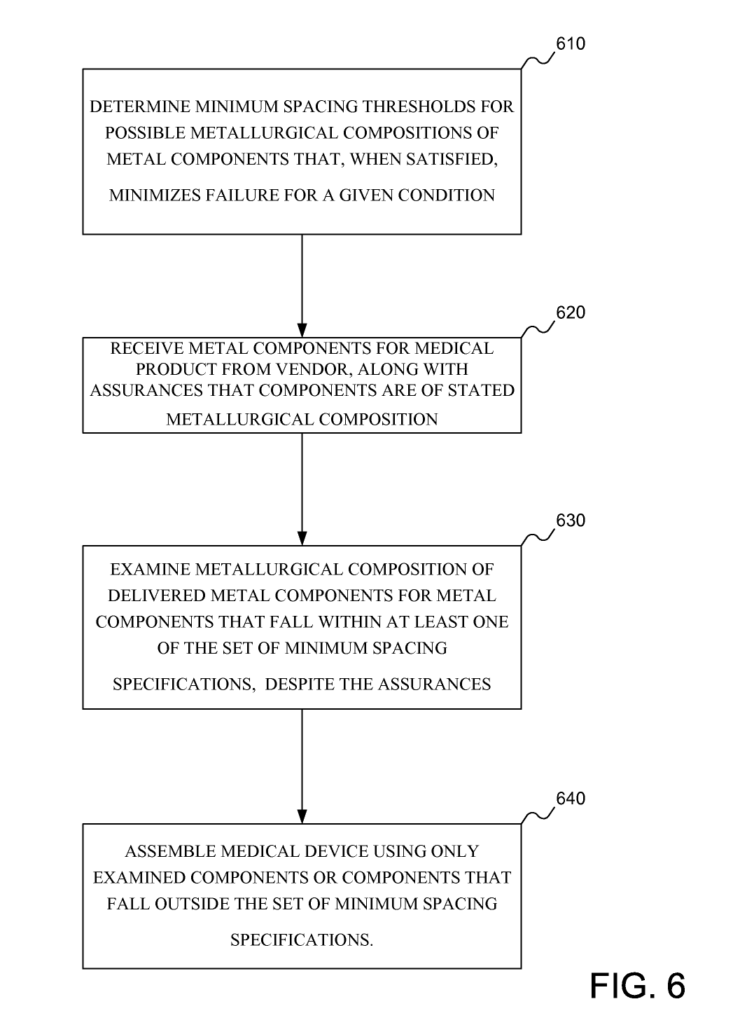

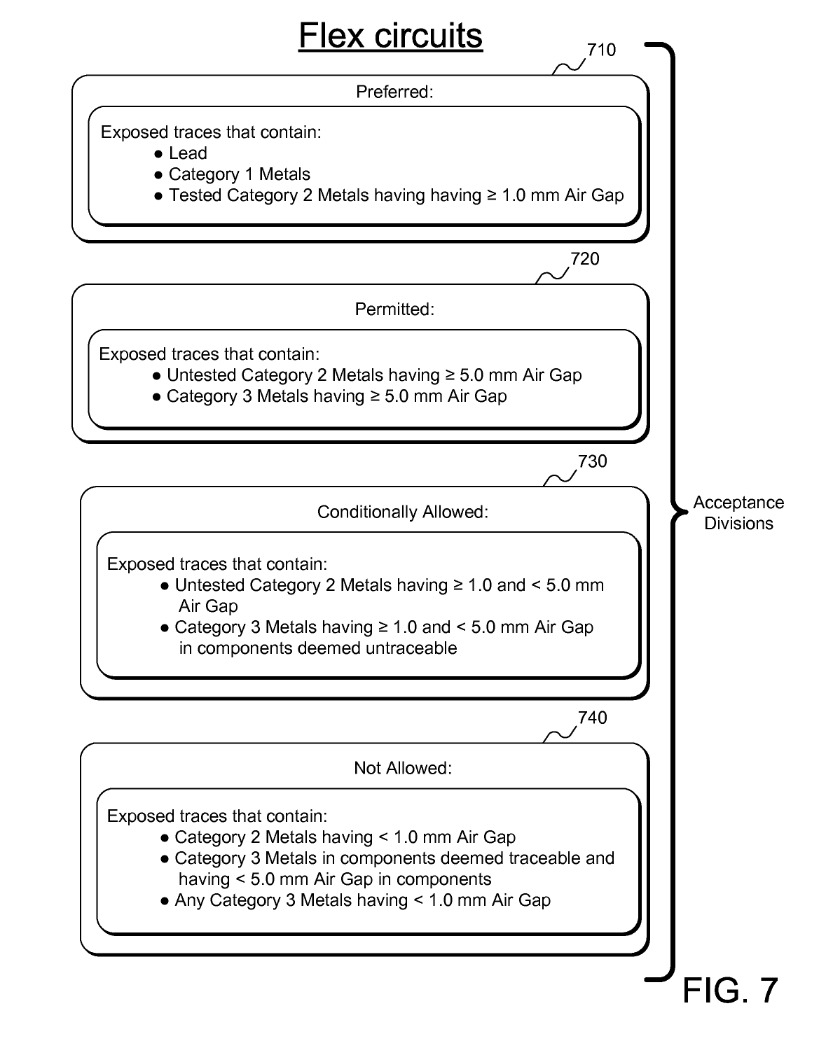

FIG. 6 is a flow diagram illustrating example methods specific to establishing a process to minimize the possible failure of a medical device by a metal whisker short. A process 610 begins by establishing a threshold measurement that minimizes or diminishes the probability of failure due to metal whiskering. As described above, components made from different materials may have different specifications. For example, components that contain Lead are at a very low risk for developing whiskers and may have a smaller air gap threshold than for other materials, such as iNemi category 1, iNemi category 2, or iNemi category 3 metals. Detailed examples are given below, with reference to FIGS. 7-10.

Some of the spacing thresholds may be based on a minimum distance between structures of the components, for example pin structures. Some threshold are based on an absolute minimum distance between pins, while other thresholds are based on average pin distance, also referred to as pitch or air gap thresholds. Other embodiments of the invention may use other spacing thresholds.

After the spacing thresholds are established in the process 610, the components including metal are received from the vendor or vendors in a process 620. Additionally received in the process 620 are assurances that the received components are of a stated metallurgical composition. These assurances may be made by product specifications, advertising material, or other written or oral assurances. The assurances may also be in the form of test data provided by the vendor of the components containing metal.

If the components received in the process 620 include spacing gaps beneath the threshold established in the process 610, then a metallurgical analysis is performed. The analysis can include XRF analysis, SEM analysis, or examining metallurgical analysis data that was produced by the vendor. Finally, in the process 640, only components that have satisfactorily passed the established tests are assembled into the final medical device.

The above description outlines general processes according to embodiments of the invention, and how to minimize the likelihood of assembling a medical device that is prone to failure for reasons of a particular defect. Described below is a detailed example of how the embodiments of the invention can be used to generate a medical device with a minimum or reduced likelihood of failing due to metal whiskering.

FIG. 7 is a block diagram illustrating various example medical device manufacturer acceptance levels for flex circuits incorporated into the medical device, according to embodiments of the invention. The medical device can be a defibrillator used for restarting someone's heart, for example.

Flex circuits are those circuits that, as compared to rigid structures, may need to be flexed, moved, or manipulated during assembly of the medical device. Some flex circuits include exposed traces, which may or may not be covered by another material. In this example components for use in the flex circuits are broken into acceptance divisions based on preference for use within the medical device. For example, with regard to circuits that include exposed traces, there are particular types of components that are in a preferred division 710, some that are permitted but not preferred 720, some that are conditionally allowed 730, and those that are not allowed 740.

The components most preferred for the flex circuits include exposed traces made from (or containing) Lead or metals found in iNemi Category 1. The International Electronics Manufacturing Initiative (iNEMI) is an organization dedicated to global electronics manufacturing. Also within the preferred division 710 are components having exposed traces made from iNemi Category 2 metals that have been tested, using the methods described above, for metallurgical makeup, provided that such traces have an air gap of greater than or equal to 1.0 mm.

The permitted division 720 of flex circuits allows for the use of untested Category 2 metals, but only if such metals are in exposed traces that have greater than or equal to a 5.0 mm air gap. This is because, given their untested status, such exposed traces may in fact contain Tin, but having such a relatively large air gap minimizes the likelihood that Tin whiskers will be problematic. Further, iNemi Category 3 metals may be used in this division, but again only for exposed traces having an air gap of greater than or equal to 5.0 mm.

The conditionally allowed division 730 includes flex circuits having exposed traces made from Category 2 metals having an air gap of between 1.0 mm and 5.0 mm. Conditional allowance means that such parts are placed on a list for design-level mitigation of the Tin whisker failure problem. Each conditionally allowed component is analyzed for risk of failure and may include a higher level of scrutiny than other components before being allowed to be produced into the medical device. In some instances, future generations of product may be re-designed to accommodate or mitigate the use of conditionally allowed components. For example if a particular conditionally allowed component is redundant, or can be protected by applying a conformal coating, it may be conditionally allowed. Other factors of design level mitigation may include insulating barriers or including electrically isolated separation pins or traces. Additionally in the conditionally allowed division 730 are those flex circuits that include exposed traces made from Category 3 metals having an air gap between 1.0 mm and 5.0 mm in components where it was decided that such components are not critical. Typically critical components are ones that are more important to the function of the device. If a part is not critical, it is a less-important part, which is why the Category 3 materials for this size air gap can be conditionally allowed.

Conversely, if the exposed trace is formed from a Category 3 metal having between 1.0 and 5.0 mm air gap in a component deemed to be a critical component, i.e., one in whose failure would cause an important function to not work then such a component is placed in the division 730 and not allowed to be assembled into the medical device. Further components falling in the not allowed division 740 include Category 2 and Category 3 metals having less than a 1.0 mm air gap.

Using the divisions 710, 720, 730, and 740 of FIG. 7 allows the manufacturer of a medical device to create a tiered approach or decision flow to determine which materials, such as metals in the flex circuits of the medical device are acceptable for that use. Specifically, first the component feature is reviewed to determine air gap spacing. If the air gap is greater than 5.0 mm, then any iNemi Category metal may be acceptable. If the air gap is less than 5.0 mm but greater than 1.0 mm, then the specific Category of the metal used in the component is ascertained and classified according to the divisions in FIG. 7. Another decision flow could instead begin with determining the metal Category in a test or based on a test report. If, for example, the metal component included an iNemi Category 1 material, then the divisions in FIG. 7 could inform the medical device manufacturer that such a component is acceptable, no matter the size of the air gap of the exposed trace for use in a flex circuit.

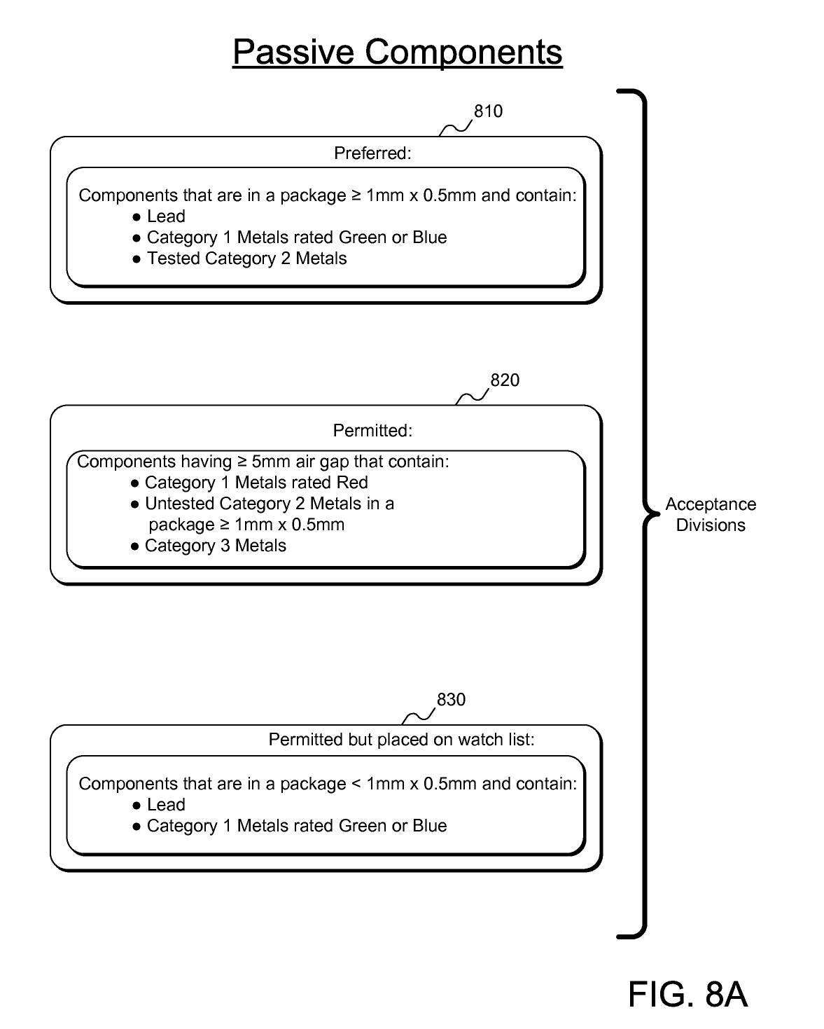

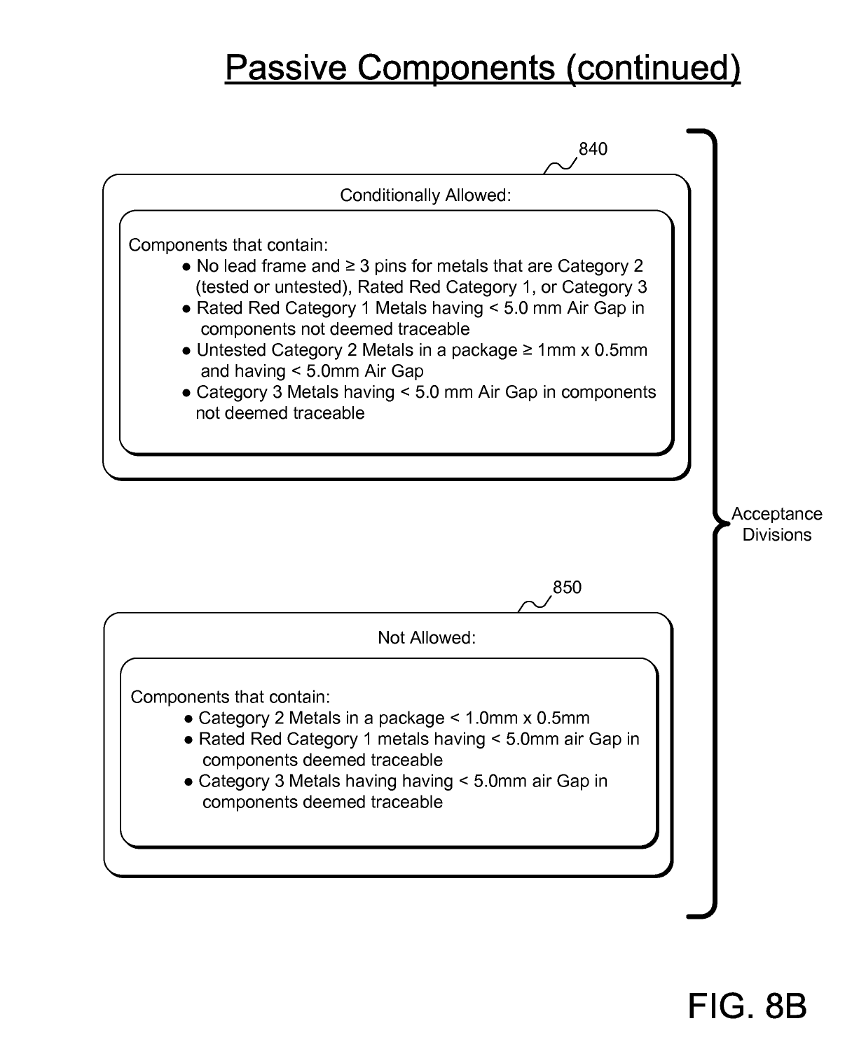

FIGS. 8A and 8B together form a block diagram illustrating various example medical device manufacturer acceptance levels of passive components incorporated into the medical device, according to embodiments of the invention. Passive components include components such as resistors, capacitors, and inductors, etc. FIGS. 8A and 8B include divisions similar to the divisions of FIG. 7, which will not be separately described for the sake of brevity. There is an additional division in FIG. 8A of “permitted but placed on watch list” 830. This division 830 is for components that do not currently contain Tin, but, if a vendor switched the metal structures within the component to Tin then the component would be at risk for causing failure of the medical device due to Tin whiskering. Thus, the medical device manufacturer can prevent such unfortunate substitution by “watching” the supply chain and the supplied components to ensure that the components are not switched to Tin without notice. Specific to this passive component list, passive components placed on the watch list 830 include components in a package smaller than 0.5 sq mm and presently made of Lead or an iNemi Category 1 Metal that is rated as Green or Blue.

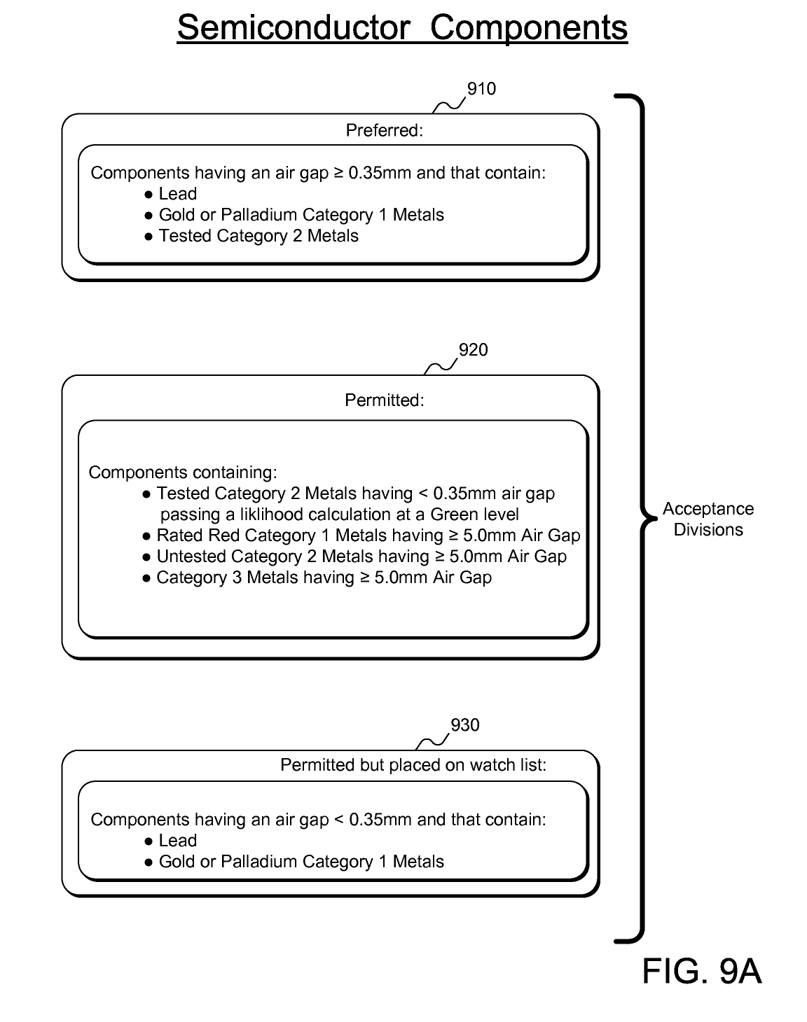

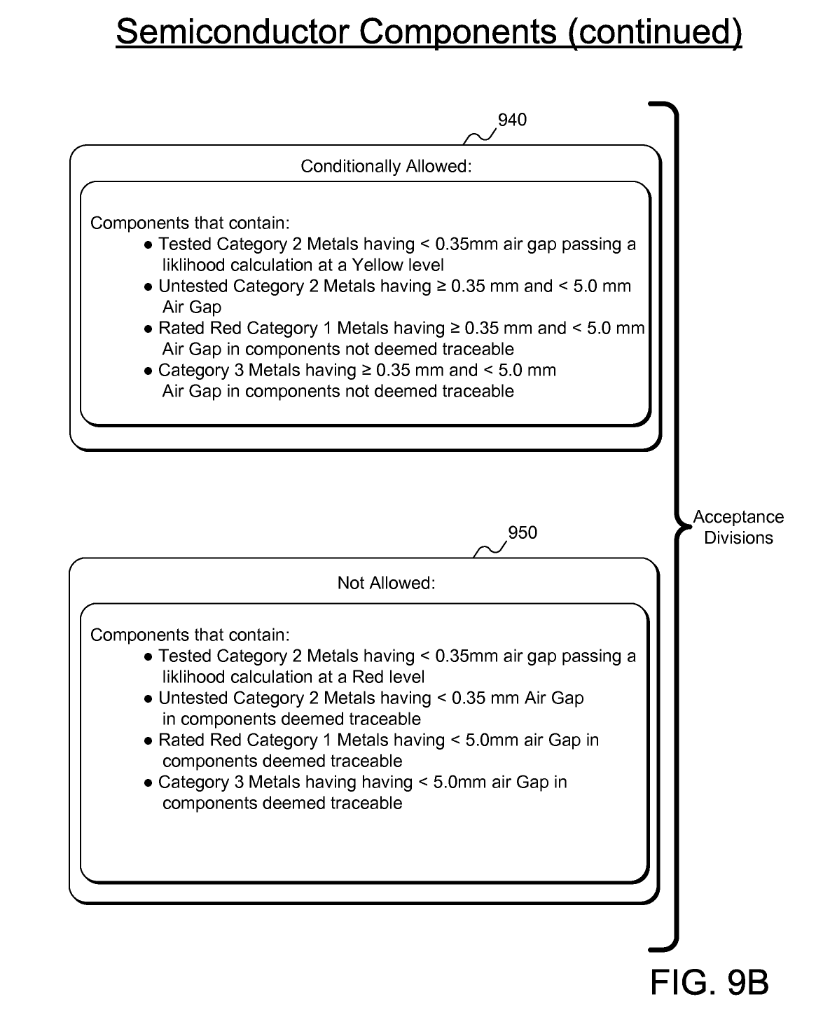

FIGS. 9A and 9B together form a block diagram illustrating various example medical device manufacturer acceptance levels of semiconductor components incorporated into the medical device, according to embodiments of the invention. Semiconductor components are those made from or including semiconductor materials, such as Class IV materials like Silicon, Germanium, etc., or Class III-V materials such as Gallium Arsenide, etc. Component divisions 910, 920, 930, 940, and 950 are similar to corresponding divisions in FIGS. 8A and 8B. The watch list division 930 of FIG. 9A includes semiconductor components having an air gap of less than 0.35 mm that contain Lead, or iNemi Category 1 materials having Gold or Palladium. Similar to the watch list 830 of FIG. 8A, the components on the watch list 930 of FIG. 9A are at risk for causing failure of the medical device due to Tin whiskers should a vendor substitute a semiconductor having less than 0.35 mm air gap spacing.

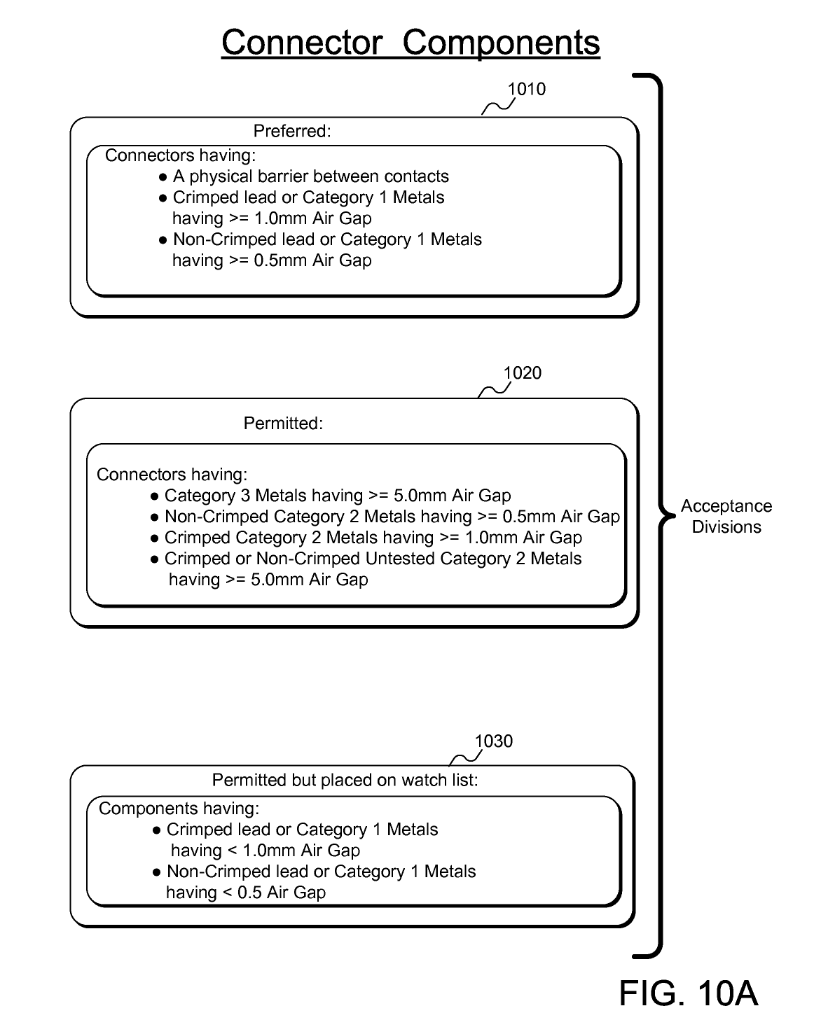

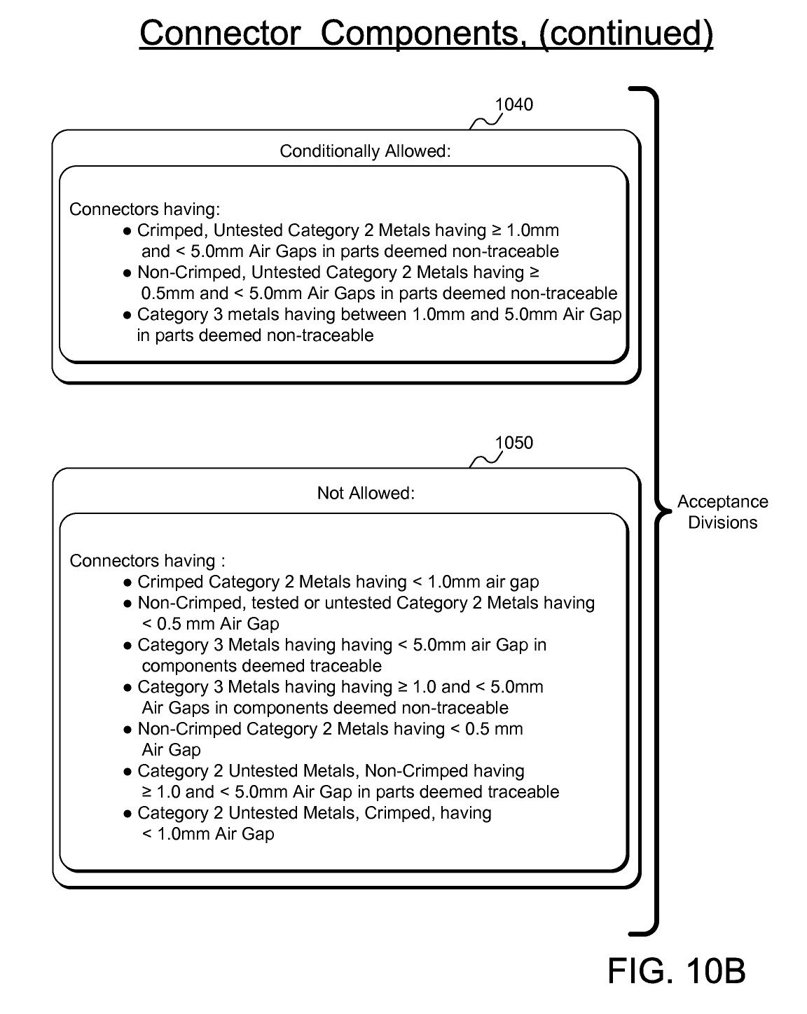

FIGS. 10A and 10B together form a block diagram illustrating various example medical device manufacturer acceptance levels of connector components incorporated into the medical device, according to embodiments of the invention. Connector components are those that electrically link one portion of the medical device to another portion and are separable. Connectors can include leads that are crimped or non-crimped. Preferred connectors are those having a physical barrier between contacts. The divisions 1010, 1020, 1030, 1040, and 1050 are similar to the corresponding divisions in both FIGS. 8A-8B, and 9A-9B, and discussion is therefore omitted for brevity.

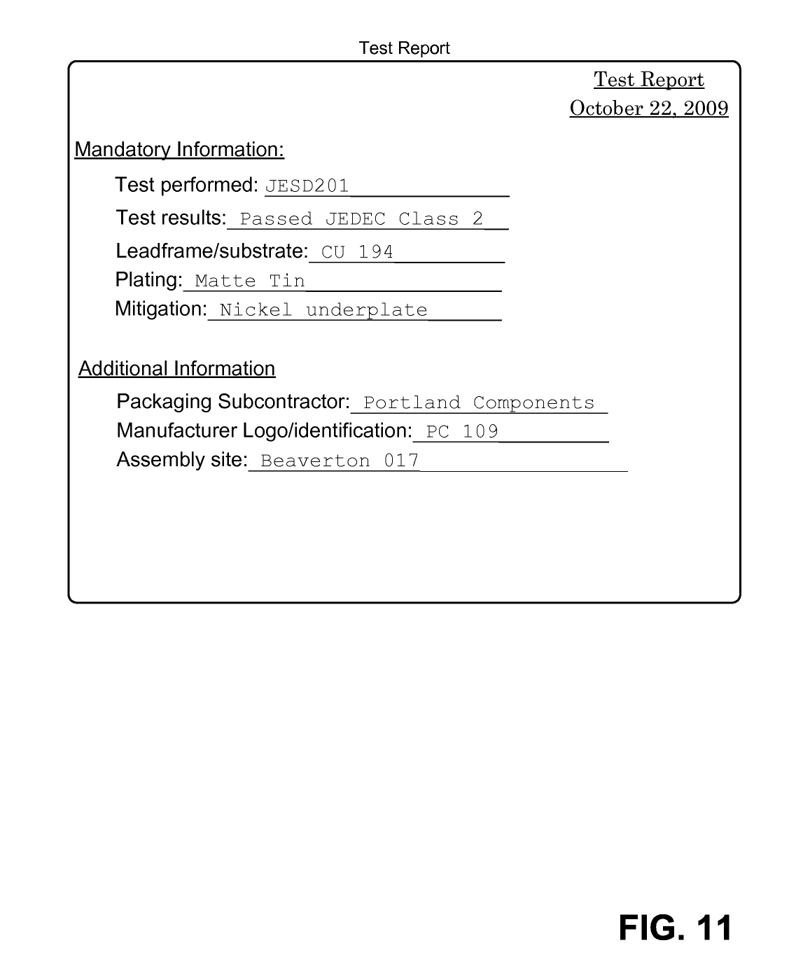

FIG. 11 is a diagram of an example test report which is one example of many processes that may be used by the medical device manufacturer to help determine whether particular lots of components or subassemblies should be included in the medical device of FIG. 2. Specifically, this is an example test report showing information that the medical device manufacturer uses to help determine whether a particular component should be allowed to be included in the assembly of the medical device. This test report may be used, for example, to help the medical device manufacturer ensure that the qualifications established in the process 430 of FIG. 4 are being satisfied in the process 440. In the report, each of the items listed under “mandatory information” is useful for the medical device manufacturer to help determine whether to include a particular component. Although the report states that such information is “mandatory,” this language reflects that it is contractually mandatory that the vendor supply this information to the medical device manufacturer, not that the medical device manufacturer must necessarily have such information to make a decision according to embodiments of the invention.

If the medical device manufacturer deems that components are unacceptable for assembly into the medical device, such components may be rejected by the medical device manufacturer before they are even shipped by the vendor. In other cases the purchasing contract may be written such that acceptance of the goods is “subject to” the components passing the specified tests, and components not passing the tests are not legally “accepted.” In yet other cases, the medical device manufacturer may be able to return non-conforming parts to the vendor, even after acceptance, based on other agreements or depending on the trade.

In some instances non-conforming goods may be put through a mitigation process to convert them into conforming goods. For example, a component having a high Tin content structure may be mitigated by dipping such a component in a solder that includes Lead. Doing so minimizes the probability of developing Tin whiskering. If the component can be modified by such a process into one that is acceptable for the medical device manufacturer, then the modified component may be assembled into the medical device.

Some embodiments of the invention have been described above, and in addition, some specific details are shown for purposes of illustrating the inventive principles. However, numerous other arrangements may be devised in accordance with the inventive principles of this patent disclosure. Further, well known processes have not been described in detail in order not to obscure the invention. Thus, while the invention is described in conjunction with the specific embodiments illustrated in the drawings, it is not limited to these embodiments or drawings. Rather, the invention is intended to cover alternatives, modifications, and equivalents that come within the scope and spirit of the inventive principles set out in the appended claims.

1. A method of making a medical device, comprising:
recognizing a possible future failure condition for a medical device;
establishing a list of components of the medical device at risk for causing the failure condition;
for components on the list, establishing one or more qualifications that if met mitigate a risk of the medical device being affected by the failure condition;
despite assurances from a vendor of the components, ensuring one or more components on the list of components satisfy the one or more qualifications; and
assembling the medical device using only components that are not on the list, or that are on the list but satisfy the one or more qualifications.

2. The method of claim 1, in which the assurances are product specifications published by the vendor.

3. The method of claim 1, in which
the failure condition is metal whiskering and
the one or more qualifications comprises satisfying a minimum dimension between metal portions of one of the components on the list.

4. The method of claim 3, in which the failure condition is tin whiskering and in which the one of the components includes a semiconductor element.

5. The method of claim 4, in which the minimum dimension is a minimum spacing of about 0.35 mm between tin portions of the semiconductor element.

6. The method of claim 1, in which at least some of the components are sourced from a vendor, the method further comprising:
rejecting the sourced components when one or more of the sourced components do not satisfy the one or more qualifications.

7. The method of claim 1, in which ensuring one or more components on the list of components satisfy the one or more qualifications is performed by an assembler of the medical device.

8. The method of claim 1, in which ensuring one or more components on the list of components satisfy the one or more qualifications is performed by a vendor of the one or more components.

9. The method of claim 1, in which ensuring one or more components on the list of components satisfy the one or more qualifications is performed by a party who is neither a vendor of the one or more components nor an assembler of the medical device.

10. The method of claim 1, in which the one or more qualifications are material type specific.

11. A medical device formed by:
recognizing a possible future failure condition for a medical device;
establishing a list of components of the medical device at risk for causing the failure condition;
for components on the list, establishing one or more qualifications that if met mitigate a risk of the medical device being affected by the failure condition;
despite assurances from a vendor of the components, ensuring one or more components on the list of components satisfy the one or more qualifications; and
assembling the medical device using only components that are not on the list, or that are on the list but satisfy the one or more qualifications.

12. The medical device of claim 11, in which the assurances are product specifications published by the vendor.

13. The medical device of claim 11, in which the failure condition is metal whiskering and in which one or more qualifications comprises satisfying a minimum dimension between metal portions of one of the components on the list.

14. The medical device of claim 13, in which the failure condition is tin whiskering and in which the one of the components includes a semiconductor element.

15. The medical device of claim 14, in which the minimum dimension is a minimum spacing of about 0.35 mm between tin portions of the semiconductor element.

16. The medical device of claim 11, in which at least some of the components are sourced from a vendor, the method further comprising:
rejecting the sourced components when one or more of the sourced components do not meet the one or more qualifications.

17. The medical device of claim 11, in which ensuring one or more components on the list of components satisfies the one or more qualifications is performed by a manufacturer of the medical device.

18. The medical device of claim 11, in which ensuring one or more components on the list of components satisfies the one or more qualifications is performed by a vendor of the one or more components.

19. The medical device of claim 11, in which ensuring one or more components on the list of components satisfies the one or more qualifications is performed by a party who is neither a vendor nor the assembler of the medical device.

20. The medical device of claim 11, in which the one or more qualifications changes based on component material type.

21. A method of building a medical product conforming to a design, comprising:
determining a set of respective minimum spacing specifications for a set of possible metallurgical compositions of metal components that, when such set of specifications are satisfied, substantially minimizes the probability of failure for a given condition;
receiving metal components for the medical product from a vendor, along with assurances that the components are of a stated metallurgical composition;
for metal components that fall within at least one of the set of minimum spacing specifications, examining a metallurgical composition of the received metal components, despite the assurances; and
assembling the medical product using only the examined components or components that fall outside the set of minimum spacing specifications.

22. The method of claim 21, in which the assurances are product data specifications published by the vendor.

23. The method of claim 21, in which the assurances state that the metal components contain lead.

24. The method of claim 21, in which at least one of the possible metallurgical compositions is tin, and in which the respective minimum spacing specification is a 0.35 mm air gap between tin contacts of a semiconductor device.

25. The method of claim 21, in which at least one of the possible metallurgical compositions is lead, and in which the respective nominal spacing specification is a 1.0 mm or greater spacing in a crimped connector.

26. The method of claim 21, further comprising generating a watch list for at least some of the metal components.

27. The method of claim 21, in which examining a metallurgical composition of the received metal components is performed by a manufacturer of the medical product.

28. The method of claim 21, in which examining a metallurgical composition of the received metal components is performed by a vendor of the received metal components.
